(19) United States
(12) Patent Application Publication
(10) Pub. No.: US 2026/0000379 A1
(43) Pub. Date: Jan. 1, 2026

(54) Positioning Scan Method and Apparatus, Dual-Source CT Apparatus, Medium and Product

(71) Applicant: Siemens Healthineers AG, Forchheim (DE)

(72) Inventors: Lei Min Shang, Shanghai (CN); Jing Bo Wang, Shanghai (CN)

(73) Assignee: Siemens Healthineers AG, Forchheim (DE)

(21) Appl. No.: 19/252,001

(22) Filed: Jun. 27, 2025

(30) Foreign Application Priority Data
Jun. 27, 2024 (CN) 202410854123.5

(51) Int. Cl. A61B 6/00; A61B 6/03; A61B 6/40

(52) U.S. Cl. CPC A61B 6/547; A61B 6/03; A61B 6/4007; A61B 6/5205; A61B 6/5211; A61B 6/542

(57) ABSTRACT

A positioning scan method and apparatus for dual-source CT systems addresses the low working efficiency of existing positioning scan methods. The system may include first and second X-ray tubes that automatically return to predetermined initial positions before positioning scans: the first tube positioned directly above or below the gantry, and the second tube positioned at a lateral location on the gantry. The positioning scan method receives positioning scan instructions and executes operations based on instruction type. For front image scan instructions, the first tube may perform exposure and obtain front projection data. For lateral image scan instructions, the second tube may perform exposure and obtain lateral projection data. For dual-positioning image scan instructions, both tubes may perform exposure, obtaining both front and lateral projection data concurrently. This improves operational efficiency by eliminating tube repositioning delays and enabling simultaneous dual-plane imaging, thereby reducing overall scan time and enhancing workflow.

### CROSS REFERENCE TO RELATED APPLICATIONS

This patent application claims priority to Chinese Patent Application No. 202410854123.5, filed Jun. 27, 2024, which is incorporated herein by reference in its entirety.

### BACKGROUND

### Field

The present disclosure relates to the technical field of medical equipment, in particular to a positioning scan method and apparatus, a dual-source CT apparatus, a non-transitory computer-readable storage medium and a computer program product.

### Related Art

Computed Tomography (CT) technology is an examination technique that is commonly used in many fields, such as medicine. A CT apparatus is a complex diagnostic system consisting of many high-precision electrical components, mainly a tube for emitting rays, a detector, a calibrator and a high-voltage generator. A common CT apparatus is typically equipped with a ray tube system and a detector system (i.e. single-source CT), and thereby acquires an image of a human body. To increase temporal resolution and further improve the quality of CT images, dual-source CT (dual-source computed tomography, DSCT) has also been proposed at the present time. Dual-source CT is a CT apparatus that uses two ray tube systems and two detector systems to acquire images of the human body simultaneously.

To perform scanning and imaging of the human body using the CT apparatus, it is necessary to place the examination subject on a bed board of a patient examination table, and move the bed board to deliver the examination subject in a horizontal state into the scanning region of the CT gantry, and only then can the scanning examination be performed. The scanning examination generally includes two scanning stages: the first stage is a positioning scan (topo scan), i.e. a stage of determining the scanning range, in which the start position, scan length and dosage scheme of a tomography scan (tomo scan) are determined according to the acquired positioning scan image. The second stage is the tomography scan, i.e. the stage of actual scanning and imaging. In the CT apparatus, the positioning scan image is also called the topo scan image.

When performing the positioning scan in the first stage, the positioning scan image required to determine the scanning range of the target organ might be different for different pre-scanned target organs or application scenarios. For example, sometimes, it might only be necessary to acquire a front positioning scan image or a lateral positioning scan image; at other times, it might be necessary to acquire a front positioning scan image and a lateral positioning scan image simultaneously (i.e. dual positioning scan images).

However, existing positioning scan methods for dual-source CT apparatuses are afflicted by the problem of low working efficiency.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIGS. 1A and 1B are schematic drawings of front image scanning and lateral image scanning, respectively, of an example positioning scan method.

FIG. 2 is a flow chart of a positioning scan method according to one or more exemplary embodiments of the present disclosure.

FIGS. 3A and 3B are schematic drawings of lateral image scanning and dual positioning image scanning, respectively, by the positioning scan method according to one or more exemplary embodiments of the present disclosure.

FIG. 4 is a structural block diagram of a positioning scan apparatus according to one or more exemplary embodiments of the present disclosure.

FIG. 5 is a structural block diagram of a dual-source CT apparatus according to one or more exemplary embodiments of the present disclosure.

The exemplary embodiments of the present disclosure will be described with reference to the accompanying drawings. Elements, features and components that are identical, functionally identical and have the same effect are—insofar as is not stated otherwise—respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software or a combination of hardware and software.

An object of the present disclosure is to provide a positioning scan method and apparatus, a dual-source CT apparatus, a non-transitory computer-readable storage medium and a computer program product, for the purpose of increasing the scanning efficiency of a dual-source CT apparatus in a positioning scan stage.

To achieve the above object, a first aspect of the present disclosure provides a positioning scan method, applied to a dual-source CT apparatus, wherein a first tube and a second tube in the dual-source CT apparatus have automatically returned to initial positions before a positioning scan is performed, and wherein an initial position of the first tube is located directly above/below a gantry, and an initial position of the second tube is located at a left/right lateral position on the gantry; the positioning scan method may comprise: receiving a positioning scan instruction; and performing the following operations according to a type of the positioning scan instruction: if it is a front image scan instruction, controlling the first tube to perform exposure, to obtain first front projection data; if it is a lateral image scan instruction, controlling the second tube to perform exposure, to obtain first lateral projection data; if it is a dual positioning image scan instruction, controlling the first tube and the second tube to simultaneously perform exposure, to obtain second front projection data and second lateral projection data respectively.

This technical solution optimizes positioning scan logic in the dual-source CT apparatus by incorporating the second tube in the topo scan, reducing the duration of a tube movement process, and thereby reducing the waiting time of the positioning scan as a whole, and increasing the overall scanning efficiency.

In an exemplary embodiment, the positioning scan method may further comprise: subjecting the second front projection data and the second lateral projection data to interference correction, to obtain the second front projection data and the second lateral projection data in corrected form; obtaining a second front positioning scan image by reconstruction on the basis of the second front projection data in corrected form, and obtaining a second lateral positioning scan image by reconstruction on the basis of the second lateral projection data in corrected form. This technical solution can eliminate projection data errors caused by overlapping of rays.

In an exemplary embodiment, the positioning scan method may further comprise subjecting the second lateral positioning scan image to field-of-view expansion correction, to enable it to be matched with the second front positioning scan image. This technical solution can avoid a situation where the second lateral positioning scan image has incomplete field-of-view coverage and is thus unable to be matched with the second front positioning scan image.

In an exemplary embodiment, the positioning scan method may further comprise obtaining a first front positioning scan image by reconstruction according to the first front projection data. This technical solution can determine a start position, a scan length and a dosage scheme of a subsequent Tomo scan according to the first front positioning scan image.

In an exemplary embodiment, the positioning scan method may further comprise obtaining a first lateral positioning scan image by reconstruction according to the first lateral projection data. This technical solution can determine a start position, a scan length and a dosage scheme of a subsequent Tomo scan according to the first lateral positioning scan image.

In an exemplary embodiment, when performing a dual positioning image scan, a first dose is used during exposure by the first tube, a second dose is used during exposure by the second tube, and the second dose is less than the first dose. This technical solution can reduce harm to the examination subject.

A second aspect of the present disclosure provides a dual-source CT apparatus, comprising: a first tube and a second tube, which have automatically returned to initial positions before a positioning scan is performed, wherein an initial position of the first tube is located directly above/below a gantry, and an initial position of the second tube is located at a left/right lateral position on the gantry; and a controller, configured to perform the method described in the present disclosure.

A third aspect of the present disclosure provides a non-transitory computer-readable storage medium storing a computer program, wherein the computer program, when executed by a processor, realizes the method described in the present disclosure.

A fourth aspect of the present disclosure provides a computer program product, comprising a computer program which, when executed by a processor, realizes the method described in the present disclosure.

In summary, compared with compared with conventional approaches, the positioning scan method and apparatus, the dual-source CT apparatus, the non-transitory computer-readable storage medium and the computer program product provided in the present disclosure have the following beneficial effects:

When a front image scan instruction is received, exposure is performed by the first tube located directly above/below the gantry; when a lateral image scan instruction is received, exposure is performed by the second tube located at the left/right lateral position on the gantry; when a dual positioning image scan instruction is received, exposure is performed simultaneously by the first tube located directly above/below the gantry and the second tube located at the left/right lateral position on the gantry. Compared with conventional approaches, when performing a lateral image scan and a dual positioning image scan, there is no need for the first tube to move to a corresponding position, so the waiting time of the scan as a whole is reduced, thus increasing the overall positioning scan efficiency. Especially in the case of a dual positioning image scan, it is possible to obtain front projection data and lateral projection data simultaneously in a single exposure, significantly reducing the duration of scanning, and thus increasing the competitiveness of the dual-source CT apparatus in high-throughput clinical scenarios.

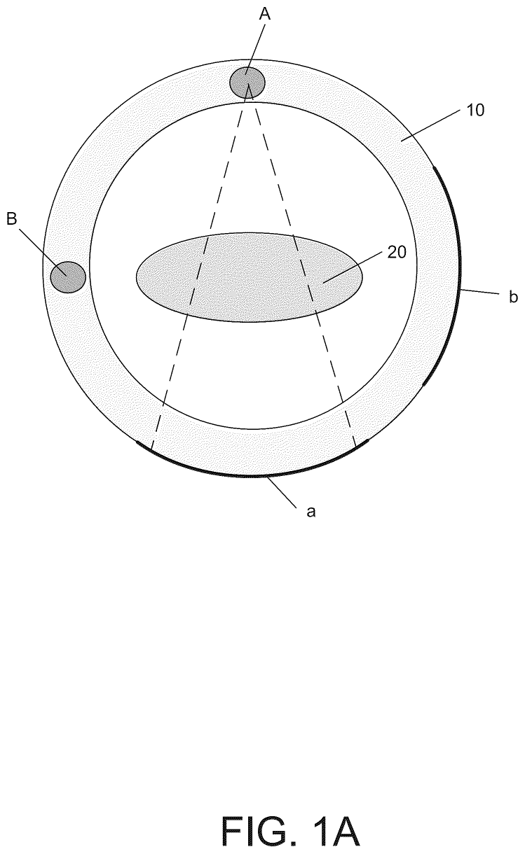

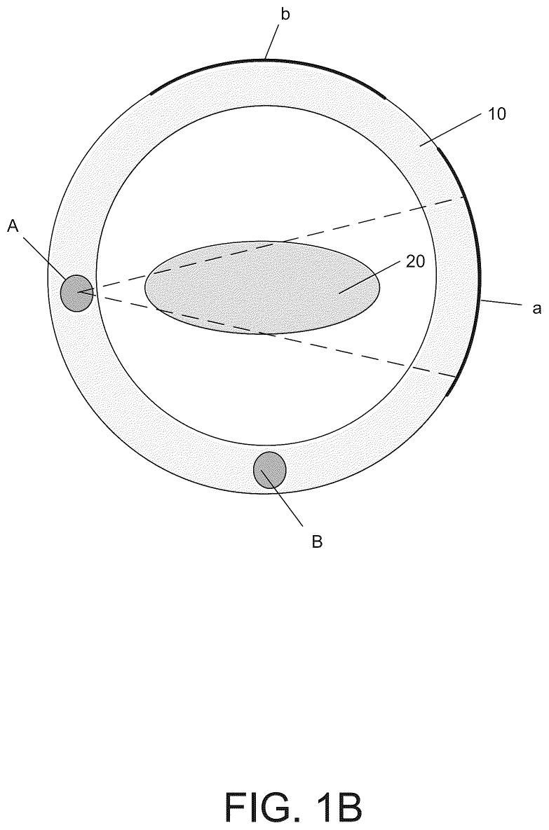

Existing positioning scan methods for dual-source CT apparatuses use only one of the tubes (i.e. tube A located directly above the gantry 10 in FIGS. 1A and 1B) to perform exposure, so as to obtain a front positioning scan image, a lateral positioning scan image and dual positioning scan images of the examination subject 20, specifically as follows:

As shown in FIG. 1A, when only a front positioning scan image is being acquired, tube A is located directly above the gantry to perform exposure, a detector a receives rays of tube A to obtain front projection data, and a front positioning scan image is subsequently obtained through image reconstruction.

As shown in FIG. 1B, when only a lateral positioning scan image is being acquired, tube A moves to a lateral position on the gantry to perform exposure, detector a receives rays of tube A to obtain lateral projection data, and a lateral positioning scan image is then obtained through image reconstruction.

When dual positioning scan images are being acquired, tube A is first located directly above the gantry to perform exposure, and detector a receives rays of tube A to obtain front projection data, then tube A moves to the lateral position on the gantry to perform exposure, and detector a receives rays of tube A to obtain lateral projection data, and then a front positioning scan image and a lateral positioning scan image (i.e. dual positioning scan images) are obtained through image reconstruction.

As can be seen, if only tube A is used for the positioning scan, time is needed for tube A to move to the target position in the course of performing the lateral positioning scan and especially the dual positioning scan, resulting in a long scan waiting time and low working efficiency, which are unsuitable for high-throughput clinical scenarios (with a large number of examination subjects).

In view of the above, an embodiment of the present disclosure provides a positioning scan method, applied to a dual-source CT apparatus. It should be explained that in this embodiment, the first tube and the second tube in the dual-source CT apparatus have automatically returned to initial positions before the positioning scan is performed; the initial position of the first tube is located directly above/below the gantry, and the initial position of the second tube is located at a left/right lateral position on the gantry, wherein “directly above/below” means the 12 o'clock/6 o'clock direction in the gantry, and “left/right lateral position” means the 3 o'clock/9 o'clock direction in the gantry. The statement “the first tube and the second tube have automatically returned to initial positions before the positioning scan is performed” means that the gantry rotates so that the first tube and the second tube return to their initial positions before the end of the previous scanning sequence or the beginning of the current scan. In an embodiment, the initial positions of the two tubes are as shown in FIG. 1A: the first tube is tube A located directly above the gantry 10, and the rays which it emits during exposure are received by detector a; the second tube is tube B located at the left lateral position on the gantry 10, and the rays which it emits during exposure are received by detector b.

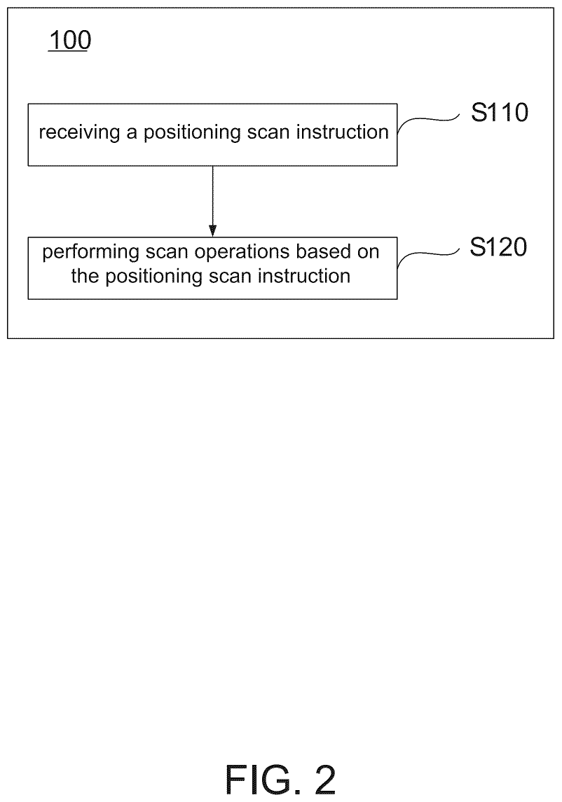

FIG. 2 shows a flow chart of a positioning scan method 100 in this embodiment. As shown in FIG. 2, the positioning scan method 100 may comprise the following steps.

Step S110, receiving a positioning scan instruction; the type of the positioning scan instruction may be a front image scan instruction, or a lateral image scan instruction, or a dual positioning image scan instruction.

Step S120, performing the following operations according to the type of the positioning scan instruction:
if it is a front image scan instruction, controlling the first tube to perform exposure, to obtain first front projection data;
if it is a lateral image scan instruction, controlling the second tube to perform exposure, to obtain first lateral projection data; and
if it is a dual positioning image scan instruction, controlling the first tube and the second tube to simultaneously perform exposure, to obtain second front projection data and second lateral projection data respectively.

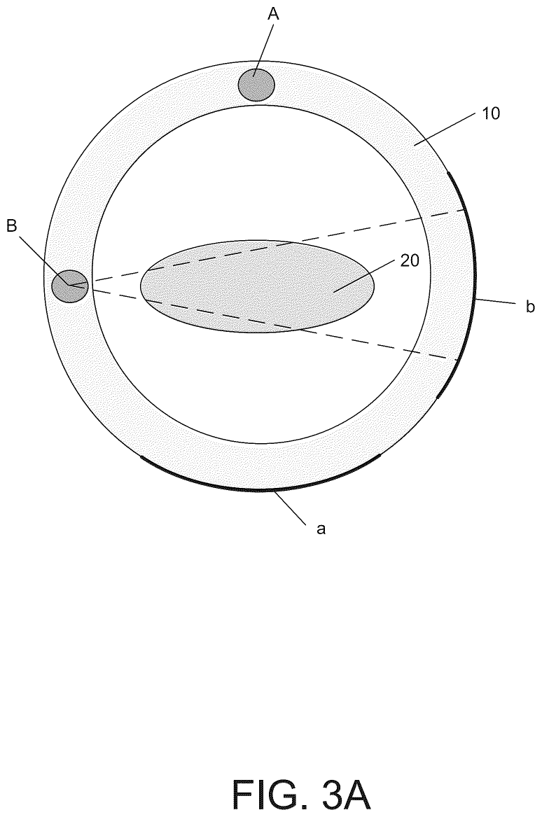

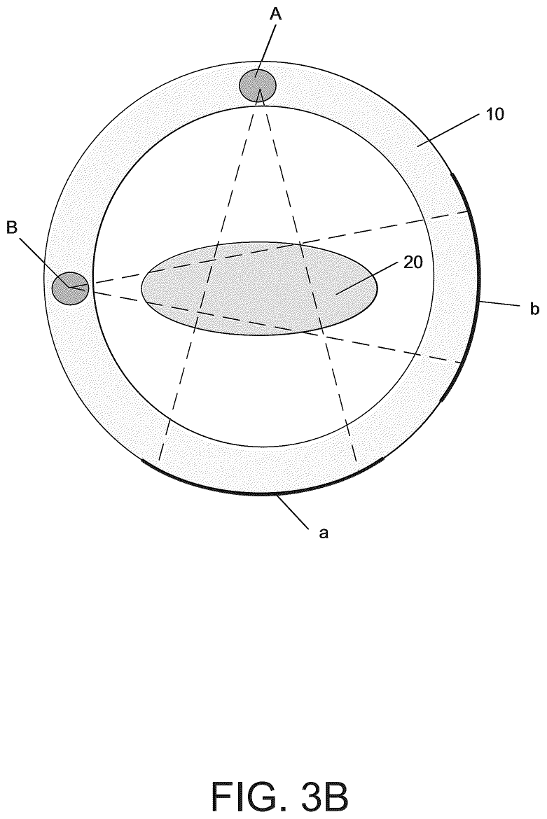

In an exemplary embodiment, when the front image scan instruction is received, exposure is performed by the first tube located directly above/below the gantry (referring to FIG. 1A, exposure is performed by tube A located directly above the gantry 10); when the lateral image scan instruction is received, exposure is performed by the second tube located at the left/right lateral position on the gantry (referring to FIG. 3A, exposure is performed by tube B located at the left lateral position on the gantry 10); when the dual positioning image scan instruction is received, exposure is performed simultaneously by the first tube located directly above/below the gantry and the second tube located at the left/right lateral position on the gantry (referring to FIG. 3B, tube A located directly above the gantry 10 and tube B located at the left lateral position on the gantry 10 perform exposure). Compared with conventional solutions, when performing a lateral image scan and a dual positioning image scan, there is no need for the first tube to move to a corresponding position, so the waiting time of the scan as a whole is reduced, thus increasing the overall positioning scan efficiency. Especially in the case of a dual-positioning image scan, it is possible to obtain front projection data and lateral projection data simultaneously in a single exposure, significantly reducing the duration of scanning, and thus increasing the competitiveness of the dual-source CT apparatus in high-throughput clinical scenarios.

In an exemplary embodiment, the positioning scan method may further comprise obtaining a first front positioning scan image by reconstruction according to the first front projection data, wherein a start position, a scan length and a dosage scheme of a subsequent Tomo scan can thus be determined according to the first front positioning scan image.

Moreover, a first lateral positioning scan image is obtained by reconstruction according to the first lateral projection data, wherein a start position, a scan length and a dosage scheme of a subsequent Tomo scan can thus be determined according to the first lateral positioning scan image.

In an exemplary embodiment, the positioning scan method may further comprise: subjecting the second front projection data and the second lateral projection data to interference correction, to obtain the second front projection data and the second lateral projection data in corrected form; obtaining a second front positioning scan image by reconstruction on the basis of the second front projection data in corrected form, and obtaining a second lateral positioning scan image by reconstruction on the basis of the second lateral projection data in corrected form. A start position, a scan length and a dosage scheme of a subsequent Tomo scan can thus be determined according to the second front positioning scan image and the second lateral positioning scan image.

It will be understood that depth positions of the first tube and the second tube in the gantry are different; “depth position” means the distance between the tube and the examination subject, i.e. the relative positions of the tube and the center of the gantry (ISO-center). As shown in FIG. 1A, tube B (i.e. the second tube) is closer to the center of the gantry 10. Moreover, there is overlapping of rays when the first tube and the second tube perform exposure simultaneously, so the second front projection data and the second lateral projection data need to be subjected to interference correction. For details of the method of interference correction, an existing algorithm may be referred to; a superfluous description is not given here.

In addition, an exemplary embodiment may further include subjecting the second lateral positioning scan image to FOV (Field Of View) expansion correction, to enable it to be matched with the second front positioning scan image. By expanding the FOV of the second lateral positioning scan image, it is possible to avoid incomplete FOV coverage in the second lateral positioning scan image, so that subsequent determination of the scanning range will not be affected. At present, examples of FOV expansion correction methods include two different types of algorithms, namely linear expansion and AI-based expansion, which are not described here superfluously.

In this embodiment, when performing a dual positioning image scan, a first dose is used during exposure by the first tube, a second dose is used during exposure by the second tube, and the second dose is less than the first dose. It will be understood that the second tube is closer to the examination subject, and its rays are attenuated less, so the exposure dose of the second tube can be less than the exposure dose of the first tube, to reduce harm to the examination subject.

According to another aspect of the present disclosure, a positioning scan apparatus applied to a dual-source CT apparatus is provided, wherein a first tube and a second tube in the dual-source CT apparatus have automatically returned to their initial positions before a positioning scan is performed, and wherein the first tube is located directly above/below a gantry, and the second tube is located at a left/right lateral position on the gantry.

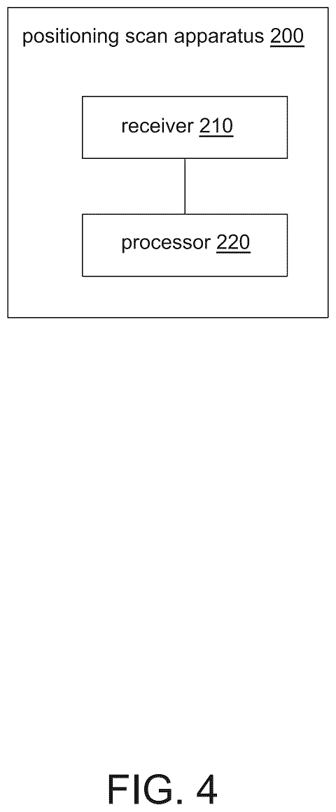

FIG. 4 shows a structural block diagram of a positioning scan apparatus 200 in this embodiment. As shown in FIG. 4, the positioning scan apparatus 200 may comprise:
an instruction receiving module (receiver) 210 configured to receive a positioning scan instruction; and
an instruction executing module (processor, controller) 220 configured to perform the following operations according to a type of the positioning scan instruction:
if it is a front image scan instruction, controlling the first tube to perform exposure, to obtain first front projection data;
if it is a lateral image scan instruction, controlling the second tube to perform exposure, to obtain first lateral projection data; and
if it is a dual positioning image scan instruction, controlling the first tube and the second tube to simultaneously perform exposure, to obtain second front projection data and second lateral projection data respectively.

It will be understood that the modules of the positioning scan apparatus 200 shown in FIG. 4 may correspond to the steps in the positioning scan method 100 described with reference to FIG. 2. Thus, the operations, features and advantages described above for the positioning scan method likewise apply to the positioning scan apparatus and the modules comprised therein. For conciseness, some operations, features and advantages are not described again here. In an exemplary embodiment, the instruction executing module (processor, controller) 220 includes processing circuitry that is configured to perform the operations and/or functions of the instruction executing module (processor, controller) 220. In an exemplary embodiment, the instruction receiving module (receiver) 210 includes processing circuitry that is configured to perform the operations and/or functions of the instruction receiving module (receiver) 210. The positioning scan apparatus 200 may further comprise a memory that stores instructions and/or data.

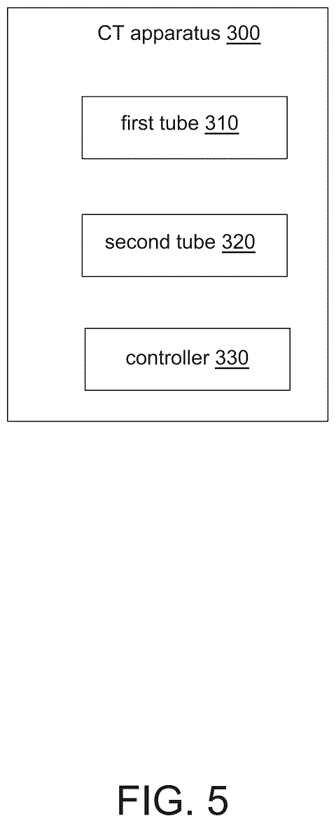

According to another aspect of the present disclosure, a dual-source CT apparatus is provided. FIG. 5 shows a structural block diagram of a dual-source CT apparatus 300 in this embodiment. As shown in FIG. 5, the dual-source CT apparatus 300 may comprise: a first tube 310 and a second tube 320, which have automatically returned to initial positions before a positioning scan is performed, wherein an initial position of the first tube 310 is located directly above/below a gantry, and an initial position of the second tube 320 is located at a left/right lateral position on the gantry; and a controller 330, the controller 330 being configured to perform the method described in the present disclosure. In an exemplary embodiment, the apparatus 300 (and/or one or more components therein, e.g., controller) includes processing circuitry that is configured to perform the operations and/or functions of the apparatus 300 (and/or respective component(s)).

The dual-source CT apparatus 300 may further comprise other components, for example, a memory that stores instructions and/or data, a collimator which blocks some of the rays emitted by the first tube 310 and the second tube 320 for the purpose of imaging a desired imaging object region, and/or a ray detector which receives rays that have passed through the imaging object for the purpose of imaging the imaging object.

According to another aspect of the present disclosure, a non-transitory computer-readable storage medium storing a computer program is provided, wherein the computer program, when executed by a processor, realizes the method described in the present disclosure.

According to another aspect of the present disclosure, a computer program product (e.g., a computer readable medium) is provided, comprising a computer program, wherein the computer program, when executed by a processor, realizes the method described in the present disclosure.

The above exemplary embodiments of the present disclosure are not intended to limit the present disclosure. Any amendments, equivalent substitutions or improvements etc. made within the spirit and principles of the present disclosure shall be included in the scope of protection thereof.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

The various components described herein may be referred to as “modules,” “units,” or “devices.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently or configured to execute instructions or computer programs that are stored on a suitable computer-readable medium. Regardless of the particular implementation, such modules, units, or devices, as applicable and relevant, may alternatively be referred to herein as “circuitry,” “controllers,” “processors,” or “processing circuitry,” or alternatively as noted herein.

For the purposes of this discussion, the term “processing circuitry” shall be understood to be circuit(s) or processor(s), or a combination thereof. A circuit includes an analog circuit, a digital circuit, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

### REFERENCE LIST

10 Gantry
20 Examination subject
A, B Tubes
a, b Detectors
100 Positioning scan method
S110, S120 Steps
200 Positioning scan apparatus
210 Instruction receiving module (receiver)
220 Instruction executing module (processor)
300 Dual-source CT apparatus
310 First tube
320 Second tube
330 Controller

1. A positioning scan method for a dual-source Computed Tomography (CT) apparatus including a first tube and a second tube, the tubes being adapted to automatically return to initial positions before performance of a positioning scan, an initial position of the first tube being located directly above or below a gantry, and an initial position of the second tube being located at a left or right lateral position on the gantry, the positioning scan method comprising:
receiving a positioning scan instruction; and
controlling one or more of the tubes based on the positioning scan instruction, wherein:
in response to the positioning scan instruction being a front image scan instruction, controlling the first tube to perform exposure to obtain first front projection data;
in response to the positioning scan instruction being a lateral image scan instruction, controlling the second tube to perform exposure to obtain first lateral projection data; and
in response to the positioning scan instruction being a dual positioning image scan instruction, controlling the first tube and the second tube to simultaneously perform exposure to obtain second front projection data and second lateral projection data, respectively.

2. The positioning scan method as claimed in claim 1, further comprising:
performing interference correction on the second front projection data and the second lateral projection data to obtain corrected second front projection data and corrected second lateral projection data;
obtaining a second front positioning scan image by reconstruction, based on the corrected second front projection data; and
obtaining a second lateral positioning scan image by reconstruction based on the corrected second lateral projection data.

3. The positioning scan method as claimed in claim 2, further comprising: performing field-of-view expansion correction on the second lateral positioning scan image to enable a matching of the second lateral positioning scan image with the second front positioning scan image.

4. The positioning scan method as claimed in claim 1, further comprising: obtaining a first front positioning scan image by reconstruction based on the first front projection data.

5. The positioning scan method as claimed in claim 1, further comprising: obtaining a first lateral positioning scan image by reconstruction based on the first lateral projection data.

6. The positioning scan method as claimed in claim 1, wherein a dual positioning image scan comprises using a first dose during exposure by the first tube and using a second dose during exposure by the second tube, the second dose being less than the first dose.

7. A dual-source Computed Tomography (CT) apparatus comprising:
first and second tubes configured to automatically return to initial positions before performance of a positioning scan, wherein an initial position of the first tube is located directly above or below a gantry, and an initial position of the second tube is located at a left or right lateral position on the gantry; and
a controller configured to control one or more of the first and second tubes based on positioning scan instruction, wherein:
in response to the positioning scan instruction being a front image scan instruction, the controller is configured to control the first tube to perform exposure to obtain first front projection data;
in response to the positioning scan instruction being a lateral image scan instruction, the controller is configured to control the second tube to perform exposure to obtain first lateral projection data; and
in response to the positioning scan instruction being a dual positioning image scan instruction, the controller is configured to control the first tube and the second tube to simultaneously perform exposure to obtain second front projection data and second lateral projection data, respectively.

8. The dual-source CT apparatus as claimed in claim 7, wherein the controller is configured to:
perform interference correction on the second front projection data and the second lateral projection data to obtain corrected second front projection data and corrected second lateral projection data;
perform reconstruction, based on the corrected second front projection data, to obtain a second front positioning scan image; and
perform reconstruction, based on the corrected second lateral projection data, to obtain a second lateral positioning scan image.

9. The dual-source CT apparatus as claimed in claim 8, wherein the controller is configured to: perform field-of-view expansion correction on the second lateral positioning scan image to enable a matching of the second lateral positioning scan image with the second front positioning scan image.

10. The dual-source CT apparatus as claimed in claim 7, wherein the controller is configured to perform reconstruction, based on the first front projection data, to obtain a first front positioning scan image.

11. The dual-source CT apparatus as claimed in claim 7, wherein the controller is configured to perform reconstruction, based on the first lateral projection data, to obtain a first lateral positioning scan image.

12. The dual-source CT apparatus as claimed in claim 7, wherein a dual positioning image scan comprises using a first dose during exposure by the first tube and using a second dose during exposure by the second tube, the second dose being less than the first dose.

13. At least one non-transitory computer-readable medium comprising instructions stored thereon, that when executed by one or more processors associated with a dual-source Computed Tomography (CT) apparatus, cause the one or more processors to perform a positioning scan method, wherein the apparatus includes a first tube and a second tube, the tubes being adapted to automatically return to initial positions before performance of a positioning scan, an initial position of the first tube being located directly above or below a gantry, and an initial position of the second tube being located at a left or right lateral position on the gantry, the positioning scan method comprising:
receiving a positioning scan instruction; and
controlling one or more of the tubes based on the positioning scan instruction, wherein:
in response to the positioning scan instruction being a front image scan instruction, controlling the first tube to perform exposure to obtain first front projection data;
in response to the positioning scan instruction being a lateral image scan instruction, controlling the second tube to perform exposure to obtain first lateral projection data; and
in response to the positioning scan instruction being a dual positioning image scan instruction, controlling the first tube and the second tube to simultaneously perform exposure to obtain second front projection data and second lateral projection data, respectively.

14. The at least one non-transitory computer-readable medium as claimed in claim 13, wherein the positioning scan method further comprises:
performing interference correction on the second front projection data and the second lateral projection data to obtain corrected second front projection data and corrected second lateral projection data;
obtaining a second front positioning scan image by reconstruction, based on the corrected second front projection data; and
obtaining a second lateral positioning scan image by reconstruction based on the corrected second lateral projection data.

15. The at least one non-transitory computer-readable medium as claimed in claim 14, wherein the positioning scan method further comprises: performing field-of-view expansion correction on the second lateral positioning scan image to enable a matching of the second lateral positioning scan image with the second front positioning scan image.

16. The at least one non-transitory computer-readable medium as claimed in claim 13, wherein the positioning scan method further comprises: obtaining a first front positioning scan image by reconstruction based on the first front projection data.

17. The at least one non-transitory computer-readable medium as claimed in claim 13, wherein the positioning scan method further comprises: obtaining a first lateral positioning scan image by reconstruction based on the first lateral projection data.

18. The at least one non-transitory computer-readable medium as claimed in claim 13, wherein a dual positioning image scan comprises using a first dose during exposure by the first tube and using a second dose during exposure by the second tube, the second dose being less than the first dose.
